(19) United States
(12) Patent Application Publication
(10) Pub. No.: US 2010/0256723 A1
(43) Pub. Date: Oct. 7, 2010

(54) Prosthetic Valve With Device for Restricting Expansion

(75) Inventor: Robert Murray, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/417,899

(22) Filed: Apr. 3, 2009

(51) Int. Cl. A61F 2/06; A61F 2/24

(52) U.S. Cl. 623/1.2; 623/1.24; 623/2.1

(57) ABSTRACT

A prosthetic valve having a stent structure with a prosthetic valve component secured therein is disclosed that includes a device for restricting expansion, i.e., an expansion restrictor device, disposed at a blood inflow end of the stent structure. The expansion restrictor device defines a deployed diameter of the stent structure to prevent the prosthetic valve from being over-sized upon initial deployment and/or from continued expansion in vivo. The expansion restrictor device may be a loop of suture, flexible line, thread or cord for defining or constraining a circumference of the prosthetic valve with a loop diameter that is less than or substantially equal to a diameter of the treatment site in which the prosthetic valve is to be deployed in vivo.

### FIELD OF THE INVENTION

The invention relates generally to a prosthetic valve for replacing a native or previously implanted prosthetic valve in a non-surgical interventional procedure. More particularly, the invention relates to a prosthetic heart valve having a stent structure that is restricted or otherwise prevented from overexpansion when deployed in vivo.

### BACKGROUND OF THE INVENTION

A wide range of medical treatments are known that utilize “endoluminal prostheses.” As used herein, endoluminal prostheses are intended to mean medical devices that are adapted for temporary or permanent implantation within a body lumen, including both naturally occurring and artificially made lumens. Examples of lumens in which endoluminal prostheses may be implanted include, without limitation: arteries, veins, gastrointestinal tract, biliary tract, urethra, trachea, hepatic and cerebral shunts, and fallopian tubes.

Stent prostheses are known for implantation within a body lumen for providing artificial radial support to the wall tissue that defines the body lumen. To provide radial support to a blood vessel, such as one that has been widened by a percutaneous transluminal coronary angioplasty, commonly referred to as “angioplasty,” “PTA” or “PTCA”, a stent may be implanted in conjunction with the procedure. Under this procedure, the stent may be collapsed to an insertion diameter and inserted into the vasculature at a site remote from the diseased vessel. The stent may then be delivered to the desired treatment site within the affected vessel and deployed, by self-expansion or radial expansion, to its desired diameter for treatment.

Recently, flexible prosthetic valves supported by stent structures that can be delivered percutaneously using a catheter-based delivery system have been developed for heart and venous valve replacement. These prosthetic valves may include either self-expanding or balloon-expandable stent structures with valve leaflets disposed within the interior of the stent structure. The prosthetic valve can be reduced in diameter, by being contained within a sheath component of a delivery catheter or by crimping onto a balloon catheter, and advanced through the venous or arterial vasculature. Once the prosthetic valve is positioned at the treatment site, for instance within an incompetent native or previously implanted prosthetic valve, the stent structure may be expanded to hold the prosthetic valve firmly in place. One embodiment of a prosthetic valve having a stent structure is disclosed in U.S. Pat. No. 5,957,949 to Leonhardt et al. entitled “Percutaneous Placement Valve Stent”, which is incorporated by reference herein in its entirety.

Valvular heart disease is any disease process involving one or more of the valves of the heart, i.e., the aortic and mitral valves on the left and the pulmonary and tricuspid valves on the right. When a prosthetic valve is percutaneously delivered to replace a stenotic or insufficient heart valve, a fundamental concern is that the prosthesis be deployed as precisely as possible so as to assure proper functioning and avoid paravalvular leakage. In addition, the deployed prosthetic heart valve must be properly sized so as not to interfere with operation of the heart. For instance if the prosthetic heart valve includes a self-expanding stent-like support structure that has an expanded diameter that is either over-sized for the valve annulus in which it has been deployed and/or that continues to “grow” after implantation, the support structure of the prosthesis may exert an undesirable radial force upon the surrounding heart tissue during and/or after initial expansion. The application of such a radial force on the surrounding heart tissue by the self-expanding stent structure may inadvertently interfere with the electrical conduction system of the heart so as to cause heart block, which may cause lightheadedness, syncope (fainting), and/or palpitations in the patient. As such, a prosthetic heart valve having a stent structure that is prevented from being oversized upon deployment and from continued expansion in vivo may be a desirable addition to the art.

### BRIEF SUMMARY OF THE INVENTION

Embodiments hereof are directed to a prosthetic valve having a stent structure with a prosthetic valve component secured therein that includes a device for restricting expansion, i.e., an expansion restrictor device, disposed at a blood inflow end of the stent structure. The expansion restrictor device defines a deployed diameter of the stent structure to prevent the prosthetic valve from being over-sized upon initial deployment and/or from continued expansion in vivo. The expansion restrictor device may be a loop of suture or other thread-like structure having a loop diameter that is less than or substantially equal to a diameter of the treatment site in which the prosthetic valve is to be deployed in vivo. In an embodiment, the looped suture may be pre-knotted so that the knot may be tightened in vivo to secure a final diameter of the loop. In another embodiment, the looped suture may be tied to a preset diameter prior to introduction into the vasculature. In various embodiments hereof, the stent structure may be either self-expanding or balloon-expandable.

### BRIEF DESCRIPTION OF DRAWINGS

The foregoing and other features and advantages of the invention will be apparent from the following description of embodiments hereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a prosthetic valve in accordance with an embodiment hereof.

FIG. 1A is a top plan view of the prosthetic valve in FIG. 1 in the direction of line A-A.

FIG. 2 illustrates the prosthetic valve of FIG. 1 in a deployment configuration within a native aortic valve in accordance with an embodiment hereof.

FIG. 3 is a side view of a laid-out section of a prosthetic valve in accordance with another embodiment hereof.

FIG. 4 is a side view of a prosthetic valve in accordance with another embodiment hereof in a deployment configuration within a native aortic valve.

### DETAILED DESCRIPTION OF THE INVENTION

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician. However, when discussing positions of the delivery system and/or the prosthetic valve within the aorta proximate the heart, the terms “distal” and “proximal” are used in the following description with respect to the heart. More particularly, “distal” or “distally” are a position away from the heart and “proximal” or “proximally” are a position near or closer to the heart

The following detailed description is merely exemplary in nature and is not intended to limit the invention or the application and uses of the invention. Although the description of embodiments hereof is in the context of heart valve replacement, the invention may also be used for valve replacement in other body passageways where it is deemed useful. Furthermore, there is no intention to be bound by any expressed or implied theory presented in the preceding technical field, background, brief summary or the following detailed description.

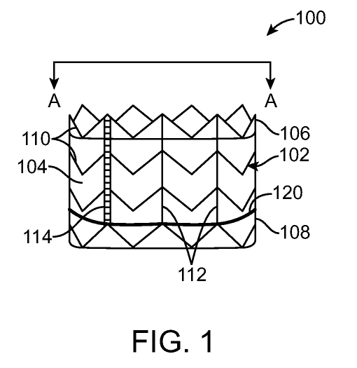

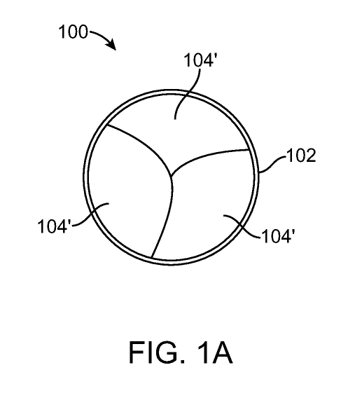

A prosthetic valve 100 in accordance with an embodiment hereof is shown and described with reference to FIGS. 1 and 1A, in which prosthetic valve 100 is shown in a deployed configuration. Prosthetic valve 100 includes a self-expanding stent structure 102 having secured therein a prosthetic valve component 104. Prosthetic valve 100 has a proximal end 106 and a distal end 108 with valve leaflets 104′ of prosthetic valve component 104 generally disposed midway therebetween. Prosthetic valve 100 is deployed and oriented relative to a direction of blood flow in vivo, such that distal end 108 defines a blood flow inlet and proximal end 106 defines a blood flow outlet with valve leaflets 104′ opening toward proximal end 106 to allow blood flow there through in an antegrade fashion.

Valve leaflets 104′ of prosthetic valve component 104 may be of a synthetic material, a xenograft natural tissue and/or a homograft natural tissue and, as shown in FIG. 1A, is a tricuspid replacement valve. In other embodiments, prosthetic valve component 104 may be a bicuspid or tubular replacement valve. Synthetic materials suitable for use in embodiments hereof include DACRON® polyester (Invista North America S.A.R.L., Wilmington, Del., U.S.A.), nylon blends, and vacuum deposition nitinol fabricated materials. Natural tissue for replacement valve leaflets may be obtained from, for example, heart valves, aortic roots, aortic walls, aortic leaflets, pericardial tissue, such as pericardial patches, bypass grafts, blood vessels, intestinal submucosal tissue, umbilical tissue and the like from humans or animals. Prosthetic valve component 104 may be sutured or otherwise securely attached within self-expanding stent structure 102 as would be known to one of ordinary skill in the art of prosthetic valve construction.

Self-expanding stent structure 102 is a tubular structure, and in the embodiment of FIG. 1, includes four wave-like or sinusoidal rings 110 attached by longitudinal connectors 112, wherein one or more longitudinal connectors 112 may be a commissure ladder 114 to which prosthetic valve component 104 is sewed or otherwise attached. Rings 110 may be attached to longitudinal connectors 112 by any attachment mechanism known to one of ordinary skill in the art of stent construction. When deployed within a native valve or previously implanted prosthetic valve, self-expanding stent structure 102 radially expands upon being released from a delivery catheter thereby providing vessel compliance and sealing of prosthetic valve 100, as well as radial support of prosthetic valve component 104. In another embodiment, self-expanding stent structure 102 may include fewer or more sinusoidal rings and have other means for attaching the sinusoidal rings together. “Self-expanding” as used herein means that the stent structures described herein have a mechanical memory or an internal restoring force to return to an expanded configuration. Mechanical memory may be imparted to a material forming the wire or tubular stent structures described herein by thermal treatment to achieve a spring temper in stainless steel, for example, or to set a shape memory in a susceptible metal alloy, such as nitinol.

In order to prevent self-expanding stent structure 102 of prosthetic valve 100 from being oversized upon deployment and/or from continuing to expand after deployment, an expansion restrictor device 120 encircles a circumference of prosthetic valve 100 proximate distal end 108. In an embodiment, expansion restrictor device 120 may be a suture or other thread-like structure that has been weaved through adjacent connectors 112 and knotted to form a loop that constrains or defines a deployed diameter of self-expanding stent structure 102. The looped suture is knotted in such a fashion that permits tightening of the knot after deployment. In such an embodiment after self-expanding stent 102 has been delivered to the treatment site and allowed to reach an expanded diameter in vivo, a knot puller/pusher device may be used to tighten the knot and secure the looped suture to a final diameter that fixes the deployed diameter of self-expanding stent structure 102. Knot puller/pusher devices as shown and described in U.S. Pat. No. 5,423,837 to Mericle et al, U.S. Pat. No. 5,693,061 to Pierce et al., U.S. Pat. No. 5,752,964 to Mericle, U.S. Pat. No. 6,511,488 to Marshall et al., and U.S. Pat. No. 7,270,672 to Singer, which are incorporated by reference herein in their entirety, may be adapted for use in embodiments hereof. In this manner, self-expanding stent structure 102 is indefinitely held at the deployed diameter and prevented from continued expansion after deployment, which avoids adversely affecting surrounding bodily structures that may be sensitive to radial pressure exerted by stent structure 102.

In embodiments hereof, expansion restrictor device 120 may be a pre-tied loop of suture, flexible line, thread or cord of a set diameter that constrains or defines a deployed diameter of self-expanding stent structure 102. In such an embodiment prior to delivery of prosthetic valve 100 to a treatment site, such as the aortic annulus when prosthetic valve 100 is a replacement aortic heart valve, a diameter of the treatment site/aortic annulus is measured via ultrasound, a CT scan or fluoroscopy and a suture, flexible line, thread or cord of a length suitable to be tied to a preset diameter that is at or slightly below the treatment site diameter is weaved around or otherwise secured to stent structure 102 and tied to form a loop with the preset diameter. Upon deployment of prosthetic valve 100 at the treatment site, the pre-tied loop of suture, flexible line, thread or cord will than fix or hold the deployed diameter of stent structure 102 at or slightly below the treatment site diameter to prevent prosthetic valve 100 from being over-sized and/or from continued expansion after deployment.

In an exemplary embodiment that represents the function of expansion restrictor devices according to embodiments hereof, self-expanding stent structure 102 may have an expanded diameter of 26 mm, for e.g., that is constrained to a deployed diameter of 25 mm, for e.g., at a blood inflow end thereof by expansion restrictor device 120. Expansion restrictor device 120 does not constrain expansion of the entire stent structure 102, such that a second deployed diameter larger than the deployed diameter of stent structure 102 at expansion restrictor device 120 occurs at least at a blood outflow end of stent structure 102. In an embodiment, the second deployed diameter may be substantially equal to the expanded diameter of self-expanding stent structure 102. Expansion restrictor device 120 continues to constrain/fix the deployed diameter of the blood inflow end of self-expanding structure 102 after initial deployment to prevent the stent structure from growing or creeping to or beyond its expanded diameter.

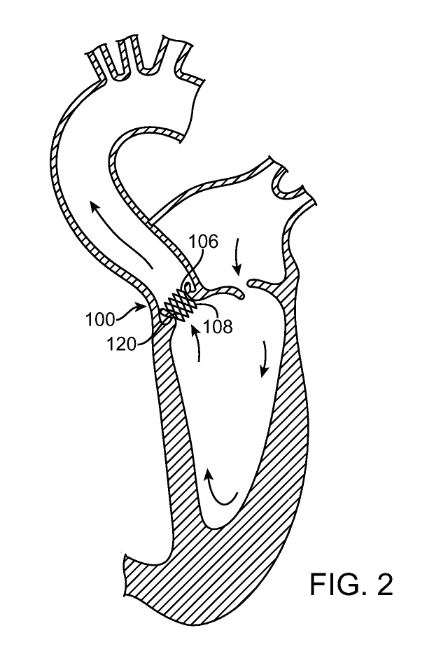

FIG. 2 illustrates prosthetic valve 100 of FIG. 1 in a deployment configuration within a native aortic valve in accordance with an embodiment hereof. Prosthetic valve 100 is shown utilized as a heart valve replacement, and more particularly as an aortic valve replacement. Blood flow is represented by the arrows shown in the figure. Prosthetic valve 100 may be delivered through the vasculature to be deployed as shown in FIG. 2 by any suitable catheter-based delivery system, such as the replacement prosthetic heart valve delivery system shown and described in U.S. Pat. Appl. Pub. No. 2008/0228254 to Ryan, which is incorporated by reference herein in its entirety. As would be known to one of ordinary skill in the art, the delivery system may have been introduced into the vasculature via a percutaneous puncture, a.k.a the Seldinger technique, or via a surgical cut-down. Methods and apparatus for accessing the arterial system with catheters and navigating such catheters to the level of the aortic arch are generally known in the art.

Prosthetic valve 100 is disposed within the native aortic valve with proximal end 106, viz., the blood flow outlet, positioned in apposition with the displaced native aortic valve leaflets and with distal end 108, viz., the blood flow inlet, concentrically disposed within the aortic annulus but spaced therefrom by expansion restrictor device 120, which constrains the deployed diameter of self-expanding stent structure 102 at distal end 108 to be less than a diameter of the opposing portion of the aortic annulus. Thus as shown in FIG. 2, stent structure 102 of prosthetic valve 100 has a deployed diameter at distal or inflow end 108 that is less than a deployed diameter of proximal or outflow end 106. In another embodiment, distal end 108 of prosthetic valve 100 may be sized by expansion restrictor device 120 to have a deployed diameter that permits contact between an outer surface of prosthetic valve distal end 108 and the opposing portion of the aortic annulus without exerting a radial force thereon.

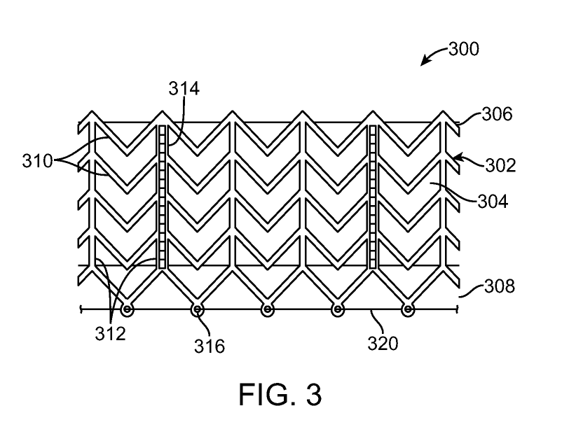

FIG. 3 is a side view of a laid-out section of a prosthetic valve 300 in accordance with another embodiment hereof. Prosthetic valve 300 includes a stent structure 302 having secured therein a prosthetic valve component 304. Prosthetic valve component 304 may be of any material or configuration as previously described above with reference to prosthetic valve component 104, and may be attached to stent structure 302 by any means known to one of ordinary skill in the art of prosthetic valve construction. Prosthetic valve 300 has a proximal end 306 and a distal end 308 with valve leaflets (not shown) of prosthetic valve component 304 generally disposed midway therebetween. Prosthetic valve 300 is deployed and oriented relative to a direction of blood flow in vivo, such that distal end 308 defines a blood flow inlet and proximal end 306 defines a blood flow outlet with the valve leaflets opening toward proximal end 306 to allow blood flow there through in an antegrade fashion.

In the embodiment of FIG. 3, tubular stent structure 302 includes a plurality of wave-like rings 310, longitudinal connectors 312 and commissure ladders 314 that are formed pre-connected as a unitary structure, such as by laser cutting or etching the entire stent body from a hollow tube or sheet. In addition, eyelets 316 are formed in the distalmost crowns or turns of stent structure 302 at prosthetic valve distal end 308. Eyelets 308 are sized to accommodate expansion restrictor device 320 therethrough, which in embodiments hereof may be a pre-knotted loop of suture, flexible line, thread or cord to be tightened and sized to a final diameter in vivo or a pre-tied loop of suture, flexible line, thread or cord having a preset diameter prior to introduction into the vasculature as described with reference to the preceding embodiments. In an embodiment, stent structure 302 may be self-expanding as described with reference to the preceding embodiment. In another embodiment, stent structure 302 may be balloon-expandable and constructed of, for e.g., platinum-iridium, cobalt chromium alloys (MP35N, L605), stainless steel, tantalum or other stent materials. Upon deployment of prosthetic valve 300 at the treatment site, expansion restrictor device 320 fixes or holds the deployment diameter of stent structure 302 at or slightly below the treatment site diameter to prevent prosthetic valve 300 from being over-sized upon initial deployment, with reference to both the balloon-expandable and self-expanding embodiments, and/or from continued in vivo expansion after deployment, with reference to the self-expanding embodiment.

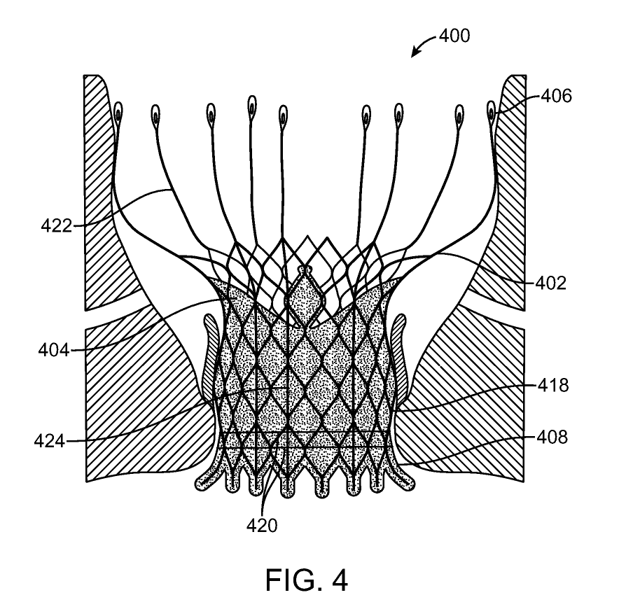

FIG. 4 is a side view of prosthetic valve 400 in accordance with another embodiment hereof in a deployment configuration within a native aortic valve. Prosthetic valve 400 includes a self-expanding stent structure 402 having secured therein a prosthetic valve component 404. Prosthetic valve component 404 may be of any material or configuration as previously described above with reference to prosthetic valve component 104, and may be attached to stent structure 402 by any means known to one of ordinary skill in the art of prosthetic valve construction. Prosthetic valve 400 has a proximal end 406 and a distal end 408 with valve leaflets (not shown) of prosthetic valve component 404 generally disposed within the portion of prosthetic valve 400 that is to be situated within the native aortic valve. As in the previous embodiments, prosthetic valve 400 is deployed and oriented relative to a direction of blood flow in vivo, such that distal end 408 defines a blood flow inlet and proximal end 406 defines a blood flow outlet with the valve leaflets opening toward proximal end 406 to allow blood flow there through in an antegrade fashion.

In the embodiment of FIG. 4, self-expanding stent structure 402 includes a tubular base portion 418 in which prosthetic valve component 404 is substantially disposed that is positioned within the aortic annulus and is shown extending into the aortic sinuses proximate the coronary arteries. An outflow portion 422 of self-expanding stent structure 402 has an expanded diameter that is greater than that of base portion 418 and a length that extends prosthetic valve 400 downstream of the sinotubular junction to anchor within the tubular portion of the ascending aorta. Stent structure base portion 418 has a diamond-shaped pattern and stent structure outflow portion 422 includes proximal segments of longitudinally extending bands 424, which are spaced apart and not covered by prosthetic valve component 404 to allow blood flow from the coronary arteries there through. As would be apparent to one of ordinary skill in the art of stent construction, self-expanding stent structure 402 having base portion 418 and outflow portion 422 may be formed from a plurality of connected stent components or as a unitary structure without departing from the scope of the present invention.

In order to prevent self-expanding stent structure 402, and more particularly tubular base portion 418, of prosthetic valve 400 from being oversized upon initial deployment and/or from continuing to expand in vivo after deployment, an expansion restrictor device 420 encircles a circumference of prosthetic valve 400 proximate distal end 408. In the embodiment of FIG. 4, expansion restrictor device 420 includes two spaced apart loops of suture, flexible line, thread or cord, which in embodiments hereof may be pre-knotted to be tightened and sized to a final diameter in vivo or pre-tied to a preset diameter prior to introduction into the vasculature as described with reference to the preceding embodiments. In another embodiment, distalmost ends of stent structure 402 located at the blood flow inlet or distal end 408 of prosthetic valve 400 may include eyelets through which expansion restrictor device 420 extends.

Prosthetic valve 400 is disposed within the native aortic valve with tubular base portion 418 positioned in apposition with the displaced native aortic valve leaflets and with distal end 408, viz., the blood flow inlet, concentrically disposed within the aortic annulus but spaced therefrom by expansion restrictor device 420, which constrains the deployed diameter of self-expanding stent structure 402 at distal end 408 to be less than a diameter of the opposing portion of the aortic annulus. In another embodiment, distal end 408 of prosthetic valve 400 may be sized by expansion restrictor device 420 to have a deployed diameter that permits contact between an outer surface of tubular base portion 418 of self-expanding stent structure 402 and the opposing portion of the aortic annulus without exerting a radial force thereon.

In each of the preceding embodiments, a suture, flexible line, thread or cord for use as an expansion restrictor device may be an elongate flexible filament of biocompatible material having sufficient strength to aid in setting the deployed diameter of the stent structure. In one embodiment, such an expansion restrictor device is a monofilament. In various other embodiments, such an expansion restrictor may be a braid of a plurality of filaments of the same or different materials. In still other embodiments, such an expansion restrictor may include a braided sheath with a single filament core, or a braided sheath with a braided core. A suture, flexible line, thread or cord for use as an expansion restrictor is constructed from a material with good tensile strength that will not stretch and/or may be pre-stressed to prevent stretching or elongation during use. Suitable biocompatible materials for such expansion restrictors include but are not limited to silk, nylon, polyethylene, and polyester, as well as other high strength materials conventionally used for sutures. In an embodiment, such expansion restrictors may include one or more pre-stretched filaments of an ultra high molecular weight polyethylene, such as a filament made from DYNEEMA fibers. Various embodiments hereof include expansion restrictors of one or more sutures, flexible lines, threads or cords having diameters in the range of 0.015 inches and 0.050 inches. However, depending on the application, one or more sutures, flexible lines, threads or cords having diameters smaller than 0.015 inches or larger than 0.050 inches may be used. Although not shown in each embodiment, expansion restrictor devices 120, 320, 420 may include one or more loops of suture, flexible line, thread or cord, which may be spaced apart as shown in the embodiment of FIG. 4 or may be in contact with each other, such as in a layered arrangement (not shown).

It will be appreciated by one of ordinary skill in the art that the stent structures shown in the preceding embodiments are merely exemplary in nature and that either self-expanding or balloon-expandable stents of various forms may be adapted for use in accordance with the teaching hereof. Some examples of stent configurations that are suitable for use in embodiments hereof are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,776,161 to Globerman, U.S. Pat. No. 5,935,162 to Dang, U.S. Pat. No. 6,090,127 to Globerman, U.S. Pat. No. 6,113,627 to Jang, U.S. Pat. No. 6,663,661 to Boneau, and U.S. Pat. No. 6,730,116 to Wolinsky et al., each of which is incorporated by reference herein in its entirety.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A prosthetic valve comprising:
a tubular stent structure having a blood inflow end and a blood outflow end;
a prosthetic valve component disposed within and secured to the stent structure, the prosthetic valve component including valve leaflets that open toward the blood outflow end of the stent structure; and
an expansion restrictor device disposed at the blood inflow end of the stent structure, wherein the expansion restrictor device defines a deployed diameter of at least the blood inflow end of the stent structure.

2. The prosthetic valve of claim 1, wherein the expansion restrictor device is a loop of suture, flexible line, thread or cord.

3. The prosthetic valve of claim 2, wherein the stent structure includes eyelets disposed around the blood inflow end through which the loop is threaded.

4. The prosthetic valve of claim 1, wherein the stent structure is self-expanding.

5. The prosthetic valve of claim 4, wherein the expansion restrictor device radially constrains the stent structure such that the deployed diameter of the blood inflow end of the stent structure is less than an expanded diameter of the stent structure.

6. The prosthetic valve of claim 5, wherein the expansion restrictor device is a loop of a non-distensible thread-like material.

7. The prosthetic valve of claim 5, wherein the blood outflow end of the stent structure has a different deployed diameter that is larger than the deployed diameter of the blood inflow end of the stent structure.

8. The prosthetic valve of claim 7, wherein the deployed diameter of the blood outflow end of the stent structure is substantially equal to the expanded diameter of the stent structure.

9. The prosthetic valve of claim 1, wherein the stent structure is balloon-expandable.

10. The prosthetic valve of claim 9, wherein the expansion restrictor device prevents over-expansion of the stent structure upon deployment of the prosthetic valve at a treatment site.

11. The prosthetic valve of claim 10, wherein the expansion restrictor device is a loop of suture, flexible line, thread or cord.

12. A method of deploying a prosthetic valve within an incompetent or insufficient heart valve, the method comprising:
positioning a prosthetic valve within the heart valve, wherein the prosthetic valve includes a tubular stent structure having secured therein a prosthetic valve component with valve leaflets and an expansion restrictor device disposed about a blood inflow end of the stent structure; and
deploying the prosthetic valve into partial apposition with the heart valve, wherein the expansion restrictor device prevents a deployed diameter of the blood inflow end of the stent structure from applying a radial force against the heart valve.

13. The method of claim 12, wherein the stent structure is self-expanding.

14. The method of claim 12, wherein the stent structure is balloon-expandable.

15. The method of claim 12, wherein the expansion restrictor device is a loop of suture, flexible line, thread or cord.

16. The method of claim 15, further comprising:
determining a diameter of the heart valve; and
sizing the loop to have a diameter that is less than the heart valve diameter such that the deployed diameter of the blood inflow end of the stent structure is less than the heart valve diameter.

17. The method of claim 16, wherein the step of sizing the loop is completed prior to the step of positioning the prosthetic valve within the heart valve.

18. The method of claim 16, wherein the step of sizing the loop is completed after the step of positioning the prosthetic valve within the heart valve.

19. The method of claim 15, further comprising:
determining a diameter of the heart valve; and
sizing the loop to have a diameter that is substantially equal to the heart valve diameter such that the deployed diameter of the blood inflow end of the stent structure is substantially equal to the heart valve diameter.
